(19) United States
(12) Patent Application Publication
(10) Pub. No.: US 2004/0105831 A1
(43) Pub. Date: Jun. 3, 2004

(54) Compositions having a pearl blend appearance additive, personal care products made therefrom

(76) Inventors: Seren Frantz, Bensalem, PA (US); Stewart Warburton, West Windsor, NJ (US)

Correspondence Address: STEVENS DAVIS MILLER & MOSHER, LLP, 1615 L STREET, NW, SUITE 850, WASHINGTON, DC, 20036, US

(21) Appl. No.: 10/638,469

(22) Filed: Aug. 12, 2003

Related U.S. Application Data
(60) Provisional application No. 60/403,241, filed on Aug. 13, 2002.

(51) Int. Cl. A61K 7/06; A61K 7/11

(52) U.S. Cl. 424/70.12

(57) ABSTRACT

Pearlizing concentrates providing pearlescence to personal care and beauty care products, such as shampoos and beauty soaps, and potentially providing added luster and sheen are disclosed. The pearlizing concentrate may include (1) a surfactant formulation, including at least one surfactant; (2) at least one pearlizing agent, such as EGDS, (3) an alkyl silicone, and (4) water. Personal care products made from this pearlizing concentrate are also disclosed.

### CROSS REFERENCE TO RELATED APPLICATIONS

This application is a non-provisional application, claiming priority from U.S. Provisional Application No. 60/403,241, filed Aug. 13, 2002, herein incorporated by reference in its entirety.

### BACKGROUND OF THE INVENTION

1. Field of the invention

The present invention

This invention relates to the addition of alkyl modified silicones which are incorporated into a cold pearl blend or concentrate to modify the appearance of the pearl crystal. The cold pearl blend concentrates are used to impart a range of specific appearances such as (1) pearl, iridescent sheen or glow, (2) opacity or (3) milky white appearance to beauty care and personal care products such as liquid soaps and shampoos.

2. Background

Cold pearlizing concentrates provide specific appearances such as pearlescence to surfactant systems, such as shampoo, body washes and hair conditioners.

Pearlescent additives, also known as pearlizing agents, are added to beauty and personal care products such as hair and skin care products to provide a pearly appearance to the products. Micron sized needles or platelets often exhibit this pearly appearance. Materials which can exhibit this effect include ethylene glycol mono-and di-stearate (EGDS and EGMS, respectively), TiO₂ coated mica, bismuth oxychloride, and natural mother of pearl. Many organic materials exhibit this pearlescence provided they can be produced in an appropriate needle or platelet shape.

Obtaining good pearlescence requires obtaining the appropriate crystal morphology. Many producers of personal care formulations have chosen to use the concentrates of pearlizing agents rather than achieving the pearlescent characteristics in-situ, i.e., during formulation blending. Utilizing a cold pearlizing concentrate allows increased consistency and reduced manufacturing time when making the finished formulation. Cold processing of the pearlizing agent additionally allows the inclusion of heat sensitive materials at any time during the formulation process. It is known in the art that using a cold pearl blend or concentrate of the appropriate appearance rather than processing the pearlizing agent in-situ is beneficial. Creating the correct pearl appearance can sometimes be difficult; however, by using appropriate crystal modifiers such as alkyl silicones crystal morphology can be controlled, allowing for control of the pearl appearance. The controlled formation of EGDS/EGMS crystals depends on two major steps of the crystallization process. The first step is the solubilization by addition of EGDS to the hot surfactant/water mixture. Good, efficient solubilization of the EGDS depends on controlled emulsification above the melting point of the crystal.

The second step is the crystallization of the pearlizing agent where shear rate and speed of cooling can play important roles. Several cooling processes are known in the art and can be used to control crystal formation. Some methods nucleate crystals in only a portion of the composition and then mix these seed crystals back into the warmer composition. A single-pass method is better for controlling crystal size since it does not require remixing crystals into the uncooled composition. Many factors, such as flow rate, temperature and time, must be monitored and controlled to achieve appropriate crystal size. A crystallization step that is not carefully controlled can, in some cases, decrease the pearlescence and the heating and cooling may have to be repeated until a satisfactory pearlescence is achieved. Although these process techniques may be used at times to control crystal formation, it is preferred to use the appropriate crystal modifiers to create the desired appearance.

### SUMMARY OF THE INVENTION

The present invention is a novel mild cold pearlizing concentrate for use in beauty and personal care compositions such as: shampoos, body wash, hand soaps, facial cleansers, conditioners, skin creams, lotions, bubble baths, liquid dishwashing products, liquid cleaners and the like. The cold pearlizing concentrate of this invention includes (1) a surfactant formulation, including at least one surfactant, (2) at least one pearlizing agent, (3) at least one alkyl silicone, and (4) water. Originally, it was believed that adding the alkyl silicones to the conventional pearl products (i.e., without any alkyl silicone) may produce a matte product. In theory, it was supposed that the alkyl chain on the silicone would associate itself with the alkyl chain on the pearlizing agent, thereby stopping the crystal growth at the points at which it came in contact with the crystallizing pearlizing agent thereby resulting in a smaller crystal size which would reduce shine, i.e., create a “matte” appearance. Surprisingly, adding the alkyl silicones maintains a high level of shine, while in some cases simultaneously increasing the opacity of finished personal care products, such as shampoos, made using the concentrate of the invention. It is also believed that the alkyl silicones actually increase crystal size. By controlling the crystal size using the alkyl silicones, the finished appearance of the concentrate can be easily controlled, whether this is creating a more “metallic” shine by increasing crystal size significantly or by creating more opacity by increasing the crystal size slightly.

In accordance with this invention, the surfactant formulation is not particularly limited. Specifically, any type of surfactant may be used with the pearlizing formulation and alkyl silicone of this invention. The invention additionally is directed to a novel method for modifying the appearance of a pearlizing agent by the addition of alkylated or esterified opacifiers, e.g., stearic ester dimethicone, which allows for the preparation of high shine and high opacity appearance.

The cold pearlizing concentrate can be a formulation of ingredients comprising: a pearlizing agent, surfactant components which may be selected from any of a wide variety of anionic, amphoteric, zwitterionic and nonionic surfactants, as well as mixtures thereof, an alkyl silicone, and water. The cold pearlizing concentrate typically can provide a variety of appearances, one of them being a brilliant sheen and pearlescence when incorporated into personal care and beauty care products.

The pearlizing formulation utilized in this invention may be the pearlizing agent disclosed by U.S. Pat. No. 6,165,955, herein incorporated by reference in its entirety.

The pearlizing agent described therein is from about 10% to about 50%, preferably from about 15% to about 40% and most preferably from about 20% to about 25%, by weight based on the total weight of the concentrate.

The pearlizing agent can typically be selected from the group consisting of polyethylene glycol mono- and di-stearates, ethylene glycol mono- and di-stearates, stearic monoethanolamide, glyceryl stearate and mixtures thereof. The preferred agents are polyethylene glycol mono- and di-stearates, and ethylene glycol mono- and di-stearates. The most preferred pearlizing agents for use are: ethylene glycol mono- and di-stearates, collectively referred to as “EGMS” or “EGDS”, respectively, herein. It is believed that any physiologically acceptable organic pearlizing agent, particularly pearlizing agents containing mono- or di- stearic alkyl chains, which could be used as a potential pearlizing agent would be useful in this invention.

The alkyl silicone utilized in this invention is a modified silicone with fatty alkyl chains grafted to the silicone backbone. Some embodiments of the invention include the alkyl silicone as a dimethiconol wax, preferably, MIRASIL WAX B (a dimethiconol behenate, available from Rhodia, Inc. of Cranbury, N.J.) or MIRASIL WAX S (a demethiconol stearate, also available from Rhodia, Inc.).

Additional background on the formulations of the invention are provided by U.S. Pat. No. 4,844,826; No. 5,925,340; No. 5,646,106; No. 5,496,544; and No. 5,560,873; as well as PCT Publication No. WO 93/17760, each of which is incorporated herein by reference in its entirety.

The cold pearlizing concentrate of this invention typically comprises 10-35%, preferably 15-30% surfactant; and 10-50%, preferably 15-40%, more preferably 20-25% pearlizing agent. The overall combination of shine and opacity of the cold pearlizing concentrate is related to the crystal size and shape of the pearlizing agent and can be modified by changes in the surfactant composition and cooling rate during crystallization.

These and other objects, features and potential advantages will become apparent to those of ordinary skill in the art from a reading of the following detailed description and the appended claims.

As used in this specification and in the appended claims, all parts, percentages, ratios and the like are by weight of active ingredient, e.g., active surfactant, unless otherwise apparent, in context, as is the case in the examples. Frequently, surfactants are sold as solutions in water or other solvents which dilute them to less than 100% active surfactant, therefore the “active surfactant” means actual amount of surfactant delivered to the composition from a commercial surfactant preparation. Thus, for example, a composition may contain 10 weight percent of a first active ingredient (on a solvent free basis), 20% of a second active ingredient (on a solvent free basis), and 20% of a third active ingredient (on a solvent free basis), and 50% water (which includes any water employed as solvent for the first, second or third active ingredients plus any added water).

All temperatures are in degrees Celsius (° C.) unless otherwise specified. All documents cited are incorporated herein by reference in their entirety. The term “cold” as utilized herein refers to the ability of the concentrate to be added without heating to the beauty and personal care products.

### DETAILED DESCRIPTION OF THE INVENTION

I. The Concentrate

A stable, mild free flowing cold pearlizing concentrate of this invention is typically prepared using (1) a surfactant formulation, comprising at least one surfactant; (2) a pearlizing agent, preferably a glycol stearate; (3) an alkyl silicone, in some embodiments an alkyl silicone wax; and (4) water. Optionally, the concentrate is provided with (5) other additives. The active surfactant generally makes up from about 10-35% of the total concentrate by weight, typically, about 15-30% and preferably about 18-25%. The pearlizing agent generally makes up about 10-50% of the total concentrate by weight, typically 15-40%, preferably 20-25%. The alkyl silicone generally makes up about 0.1-10% of the total concentrate by weight, typically 0.5-5% and preferably 2-4%. Generally, water makes up the remainder of the concentrate of the invention. In the embodiments where the concentrate includes additional additives, the amount of water is correspondingly reduced.

1. Surfactant

The surfactants of the surfactant formulation utilized in the concentrate of the invention are not limited, as any conventional surfactant may be utilized. For example, the surfactant may be ionic, anionic, zwitterionic, amphoteric, or mixtures thereof, such as those described in U.S. application Ser. No. 09/318,941, filed May 26, 1999; U.S. application Ser. No. 09/698,479, filed Oct. 30, 2000; PCT Publication No. WO 00/71651, filed May 25, 2000; U.S. application Ser. No. 09/318,942, filed May 26, 1999; U.S. application Ser. No. 09/698,149, filed Oct. 30, 2000; and PCT Publication No. WO 00/71591, filed May 25, 2000, each of which is hereby incorporated by reference in its entirety. Optional ingredients which may also be employed in the surfactant formulation or component of the present invention include, for example, preservatives, cationic surfactants, pH adjusting agents, perfumes, dyes, and sequestering agents. Preferred surfactants include alkyl/alkyl ether sulfates, cocamide MEA, betaines, amphoacetates, ethoxylated alcohols, and mixtures thereof.

A. Anionic Surfactants

Anionic surfactants may be exemplified by the alkali metal salts of organic sulfuric reaction products having in their molecular structure an alkyl radical containing from 8-22 carbon atoms and a sulfonic acid or sulfuric acid ester radical (included in the term alkyl is the alkyl portion of higher acyl radicals), as described by U.S. Pat. No. 5,925,340.

Exemplary anionic surfactants useful in the present invention are preferably selected from the group consisting of, linear alkylbenzene sulfonate, alpha olefin sulfonate, paraffin sulfonates, alkyl ester sulfonates, alkyl sulfates, alkyl alkoxy sulfate, alkyl sulfonates, alkyl alkoxy carboxylate, alkyl alkoxylated sulfates, sarcosinates, taurates, and mixtures thereof. An effective amount, typically from about 0% to about 70%, preferably about 0.1% to about 30%, more preferably from about 5 to about 20%, of anionic surfactant can be used in the present invention.

Alkyl sulfate surfactants are another type of anionic surfactant suitable for use herein. In addition to providing excellent overall cleaning ability when used in combination with polyhydroxy fatty acid amides, including good grease/oil cleaning over a wide range of temperatures, wash concentrations, and wash times, dissolution of alkyl sulfates can be obtained, as well as improved formulability in liquid detergent formulations are water soluble salts or acids of the formula ROSO₃M wherein R preferably is a C₁₀-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a cation, e.g., an alkali (Group IA) metal cation (e.g., sodium, potassium, lithium), substituted or unsubstituted ammonium cations such as methyl-, dimethyl-, and trimethyl ammonium and quaternary ammonium cations, e.g., tetramethyl-ammonium and dimethyl piperdinium, and cations derived from alkanolamines such as ethanolamine, diethanolamine, triethanolamine, and mixtures thereof, and the like. Typically, alkyl chains of C₁₂₋₁₆ are preferred for lower wash temperatures (e.g., below about 50° C.) and C₁₆₋₁₈ alkyl chains are preferred for higher wash temperatures (e.g., above about 50° C.)

Alkyl alkoxylated sulfate surfactants are another category of useful anionic surfactant. These surfactants are water soluble salts or acids typically of the formula RO(A)ₘSO₃M wherein R is an unsubstituted C₁₀-C₂₄ alkyl or hydroxyalkyl group having a C₁₀-C₂₄ alkyl component, preferably a C₁₂-C₂₀ alkyl or hydroxyalkyl, more preferably C₁₂-C₁₈ alkyl or hydroxyalkyl, A is an ethoxy or propoxy unit, m is greater than zero, typically between about 0.5 and about 6, more preferably between about 0.5 and about 3, and M is H or a cation which can be, for example, a metal cation (e.g., sodium, potassium, lithium, etc.), ammonium or substituted-ammonium cation. Alkyl ethoxylated sulfates as well as alkyl propoxylated sulfates are contemplated herein. Specific examples of substituted ammonium cations include methyl-, dimethyl-, trimethyl-ammonium and quaternary ammonium cations, such as tetramethyl- ammonium, dimethyl piperidinium and cations derived from alkanolamines, e.g., monoethanolamine, diethanolamine, and triethanolamine, and mixtures thereof. Exemplary surfactants are C₁₂-C₁₈ alkyl polyethoxylate (1.0) sulfate, C₁₂-C₁₈ alkyl polyethoxylate (2.25) sulfate, C₁₂-C₁₈ alkyl polyethoxylate (3.0) sulfate, and C₁₂-C₁₈ alkyl polyethoxylate (4.0) sulfate wherein M is conveniently selected from sodium and potassium. Surfactants for use herein can be made from natural or synthetic alcohol feedstocks. Chain lengths represent average hydrocarbon distributions, including branching.

Examples of suitable anionic surfactants are given in “Surface Active Agents and Detergents” (Vol. I and II by Schwartz, Perry and Berch). A variety of such surfactants are also generally disclosed in U.S. Pat. No. 3,929,678, issued Dec. 30, 1975 to Laughlin, et al. at Column 23, line 58 through Column 29, line 23.

B. Zwitterionic Surfactants

Zwitterionic surfactants can be exemplified by those which can be broadly described as derivatives of aliphatic quaternary ammonium, phosphonium, and sulfonium compounds in which the aliphatic radicals can be straight chain or branched and wherein one of the aliphatic substituents contains from about 8 to 18 carbon atoms and one contains an anionic water-solubilizing group such as carboxyl, sulfonate, sulfate, phosphate or phosphonate.

C. Amphoteric Surfactants

Examples of amphoterics which may be used include those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate or phosphonate.

The term “amphoteric surfactant” as utilized herein encompasses one or more amphoteric surfactants such as mixtures of amphoteric surfactants. Amphoteric surfactants known as the betaines, their derivatives, and mixtures thereof may be incorporated to provide an enhanced pearlizing effect.

Examples of suitable amphoteric surfactants include the alkali metal, alkaline earth metal, ammonium or substituted ammonium salts of alkyl amphocarboxy glycinates and alkyl amphocarboxypropionates, alkyl amphodipropionates, alkyl amphodiacetates, alkyl amphoglycinates and alkyl amphopropionates wherein alkyl represents an alkyl group having 6 to 20 carbon atoms. Other suitable amphoteric surfactants include alkyl iminopropionates, alkyl iminodipropionates and alkyl amphopropylsulfonates having between 12 and 18 carbon atoms; alkyl betaines and amidopropyl betaines and alkyl sultaines and alkylamidopropylhydroxy sultaines wherein alkyl represents an alkyl group having 6 to 20 carbon atoms.

Particularly useful amphoteric surfactants include both mono and dicarboxylates such as those of the formulae:

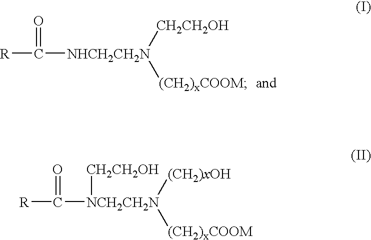

wherein R is an alkyl group of 6-20 carbon atoms, x is 1 or 2 and M is hydrogen or sodium. Mixtures of the above structures are particularly preferred.

Other formulae for the above amphoteric surfactants include the following:

Alkyl Betaines

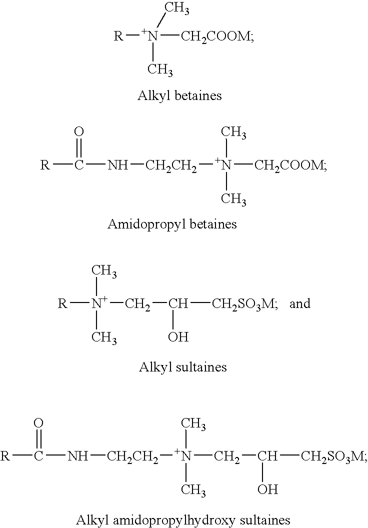

where R is a alkyl group of 6-20 carbon atoms and M is potassium, sodium or a monovalent cation.

Of the above amphoteric surfactants, particularly preferred are the alkali salts of alkyl amphodipropionates, alkyl amphodiacetates, alkyl amphoglycinates, alkyl amphopropyl sulfonates and alkyl amphopropionates wherein alkyl represents an alkyl group having 6 to 20 carbon atoms. Even more preferred are compounds wherein the alkyl group is derived from coconut oil or is a lauryl group, for example cocoamphodipropionate. Such cocoamphodipropionate surfactants are commercially sold under the trademarks MIRANOL C2M-SF CONC by Rhodia.

Other commercially useful amphoteric surfactants include: cocoamphoacetate (sold under the trademarks MIRANOL ULTRA C-32), cocoamphopropionate (sold under the trademarks MIRANOL CMSF CONC.), cocoamphodiacetate (sold under the trademarks MIRANOL C2M CONC.), lauroamphoacetate (sold under the trademarks MIRANOL HM CONC. And MIRANOL ULTRA L-32), lauroamphodiacetate (sold under the trademarks MIRANOL H2M CONC), lauroamphodipropionate (sold under the trademarks MIRANOL H2M-SF CONC), lauroamphodiacetate obtained from a mixture of lauric and myristic acids (sold under the trademark MIRANOL BM CONC.), and cocoamphopropyl sulfonate (sold under the trademark MIRANOL CS CONC.) caproamphodiacetate (sold under the trademark MIRANOL S2M CONC.), caproamphoacetate (sold under the trademark MIRANOL SM CONC.), caproamphodipropionate (sold under the trademark MIRANOL S2M-SF CONC.), and stearoamphoacetate (sold under the trademark MIRANOL DM).

Cocoamphoacetate may be present from 0% to 10% based on the total weight of the concentrate. In one embodiment, cocoamphoacetate will comprise from about 1% to about 7% and most preferably from about 2% to about 4% based on the total weight of the concentrate.

Also useful herein are the betaines and amidobetaines which are compounds of the general structure:

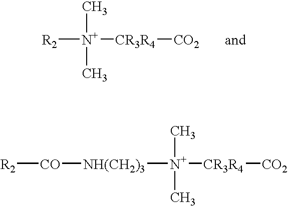

respectively wherein R₂ is C₈-C₂₂ alkyl or alkenyl; R₃ is H or C₁-C₄ alkyl; and R₄ is H or C₁-C₄ alkyl.

The betaines useful herein include the high alkyl betaines such as cocodimethyl carboxymethyl betaine, lauryl dimethyl carboxymethyl betaine, lauryl dimethyl alpha-carboxy-ethyl betaine, cetyl dimethyl carboxymethyl betaine, lauryl bis-(2-hydroxy-ethyl)carboxy methyl betaine, stearyl bis-(2-hydroxy-propyl)carboxymethyl betaine, oleyl dimethyl gamma-carboxypropyl betaine, and lauryl bis-(2-hydroxypropyl)alpha-carboxyethyl betaine. The sulfobetaines are also preferred and may be represented by cocodimethyl sulfopropyl betaine, stearyldimethyl sulfopropyl betaine, lauryl dimethyl sulfoethyl betaine, lauryl bis-(2-hydroxy-ethyl)sulfopropyl betaine and mixtures thereof. A particularly preferred composition utilizes cocoamidopropyl betaine.

In one embodiment, the amphoteric surfactant can be cocoamphoacetate and cocoamidopropyl betaine acting as amphoteric co-emulsifiers.

The amphoteric surfactant can be present from about 0% to about 35 weight percent, or 0.1 to 20 weight percent, or 4 to 16 weight percent based on the total weight of the pearlizing concentrate.

D. Nonionic Surfactants

Nonionic surfactants which may be used can be broadly defined as compounds produced by the condensation of alkylene oxide groups (hydrophilic in nature) with an organic hydrophobic compound which may be aliphatic or alkyl aromatic in nature. Particularly preferred nonionic surfactants include the sugar surfactants such as alkyl polyglycoside and polyhydroxy fatty acid amides.

This type of surfactant can function as an emulsifier and stabilizer in the formulation. The term “nonionic surfactant” as utilized herein encompasses mixtures of nonionic surfactants.

Examples of useful nonionic surfactants include condensates of ethylene oxide. These surfactants include the condensation products of primary or secondary aliphatic alcohols having from about 8 to about 24 carbon atoms, in either straight or branched chain configuration, with from about 2 to about 40, and preferably between about 2 and about 9 moles of ethylene oxide per mole of alcohol.

In one embodiment, the aliphatic alcohol comprises between about 9 and about 18 carbon atoms and is ethoxylated with between about 3 and about 12 moles of ethylene oxide per mole of aliphatic alcohol, such as the about 12 to about 15 carbon primary alcohol ethoxylates containing about 5 to about 9 moles of ethylene oxide per mole of alcohol. One such material is commercially sold under the trade name NEODOL 25-9 by Shell Chemical Company. Other commercial nonionic surfactants include NEODOL 25-6.5 and NEODOL 25-7 sold by Shell Chemical Company.

Other suitable nonionic surfactants include the condensation products of about 6 to about 12 carbon atom alkyl phenols with about 3 to about 30, and preferably between about 5 and 14 moles of ethylene oxide. Examples of such surfactants are sold under the trade manes Igepal CO 530, Igepal CO 630, Igepal CO720 and Igepal CO 730 by Rhodia. Still other suitable nonionic surfactants are described in U.S. Pat. No. 3,976,586. To the extent necessary, this patent is expressly incorporated by reference. Most preferred for use are mixed linear alcohol ethoxylates such as Laureth-7 sold as RHODASURF L-790 by Rhodia.

Suitable nonionic detergent surfactants are generally disclosed in U.S. Pat. No. 3,929,678, Laughlin et al., issued Dec. 30, 1975 incorporated herein by reference. Exemplary, non-limiting classes of useful nonionic surfactants include: amine oxides, alkyl ethoxylate, alkanoyl glucose amide, alkanolamides, and mixtures thereof.

Amine oxides are semi-polar nonionic surfactants and include water-soluble amine oxides containing one alkyl moiety of from about 10 to about 18 carbon atoms and 2 moieties selected from the group consisting of alkyl groups and hydroxyalkyl groups containing from about 1 to about 3 carbon atoms; water-soluble phosphine oxides containing one alkyl moiety of from about 10 to about 18 carbon atoms and 2 moieties selected from the group consisting of alkyl groups and hydroxyalkyl groups containing from about 1 to about 3 carbon atoms; and water-soluble sulfoxides containing one alkyl moiety of from about 10 to about 18 carbon atoms and a moiety selected from the group consisting of alkyl and hydroxyalkyl moieties of from about 1 to about 3 carbon atoms.

Semi-polar nonionic surfactants include the amine oxide surfactants having the formula

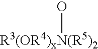

wherein R³ is an alkyl, hydroxyalkyl, or alkyl phenyl group or mixtures thereof containing from about 8 to about 22 carbon atoms; R⁴ is an alkylene or hydroxyalkylene group containing from about 2 to about 3 carbon atoms or mixtures thereof; x is from 0 to about 3; and each R⁵ is an alkyl or hydroxyalkyl group containing from about 1 to about 3 carbon atoms or a polyethylene oxide group containing from about 1 to about 3 ethylene oxide groups. The R⁵ groups can be attached to each other, e.g., through an oxygen or nitrogen atom, to form a ring structure.

These amine oxide surfactants in particular include C₁₀-C₁₈ alkyl dimethyl amine oxides and C₈-C₁₂ alkoxy ethyl dihydroxy ethyl amine oxides. Typically, the amine oxide is present in the composition in an effective amount, such as from about 0.1% to about 20%, about 0.1% to about 15%, or about 0.5% to about 10%, by weight of the total concentrate.

The polyethylene, polypropylene, and polybutylene oxide condensates of alkyl phenols, such as, the polyethylene oxide condensates, are preferred. These compounds include the condensation products of alkyl phenols having an alkyl group containing from about 6 to about 12 carbon atoms in either a straight chain or branched chain configuration with the alkylene oxide. In one embodiment, the ethylene oxide is present in an amount equal to from about 5 to about 25 moles of ethylene oxide per mole of alkyl phenol. Commercially available nonionic surfactants of this type include IGEPAL CO-630, marketed by the GAF Corporation; and TRITON X-45, X-114, X-100, and X-102, all marketed by the Rohm & Haas Company. These compounds are commonly referred to as alkyl phenol alkoxylates, (e.g., alkyl phenol ethoxylates).

The condensation products of aliphatic alcohols with from about 1 to about 25 moles of ethylene oxide. The alkyl chain of the aliphatic alcohol can either be straight or branched, primary or secondary, and generally contains from about 8 to about 22 carbon atoms. Typical are the condensation products of alcohols having an alkyl group containing from about 10 to about 20 carbon atoms with from about 2 to about 18 moles of ethylene oxide per mole of alcohol. Examples of commercially available nonionic surfactants of this type include TERGITOL 15-S-9 (the condensation product of C₁₁-C₁₅ linear secondary alcohol with 9 moles ethylene oxide), TERGITOL 24-L-6 NMW (the condensation product of C₁₂-C₁₄ primary alcohol with 6 moles ethylene oxide with a narrow molecular weight distribution), both marketed by Union Carbide Corporation; NEODOL 45-9 (the condensation product of C₁₄-C₁₅ linear alcohol with 9 moles of ethylene oxide), NEODOL 23-6.5 (the condensation product of C₁₂-C₁₃ linear alcohol with 6.5 moles of ethylene oxide), NEODOL 45-7 (the condensation product of C₁₄-C₁₅ linear alcohol with 7 moles of ethylene oxide), NEODOL 45-4 (the condensation product of C₁₄-C₁₅ linear alcohol with 4 moles of ethylene oxide), marketed by Shell Chemical Company, and KYRO EOB (the condensation product of C₁₃-C₁₅ alcohol with 9 moles ethylene oxide), marketed by The Procter & Gamble Company. Other commercially available nonionic surfactants include DOBANOL 91-8 marketed by Shell Chemical Co. and GENAPOL UD-080 marketed by Hoechst. This category of nonionic surfactant is referred to generally as “alkyl ethoxylates.”

Typical alkylpolyglycosides have the formula R²O(CₙH₂ₙO)ₜ(glycosyl)ₓ, wherein R² is selected from the group consisting of alkyl, alkyl-phenyl, hydroxyalkyl, hydroxyalkylphenyl, and mixtures thereof in which the alkyl groups contain from about 10 to about 18, preferably from about 12 to about 14, carbon atoms; n is 2 or 3, preferably 2; t is from 0 to about 10, preferably 0; and x is from about 1.3 to about 10, preferably from about 1.3 to about 3, most preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4- and/or 6-position, preferably predominantly the 2-position. The nonionic surfactant may be incorporated in the cold pearlizing concentrate in an amount of from about 3% to about 30%; preferably from about 8% to about 25% and most preferably from about 10% to 20%, based on the total weight of the concentrate.

Fatty acid amide surfactants having the formula:

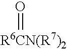

wherein R⁶ is an alkyl group containing from about 7 to about 21 (preferably from about 9 to about 17) carbon atoms, and each R⁷ is selected from the group consisting of hydrogen, C₁-C₄ alkyl, C₁-C₄ hydroxyalkyl, and —(C₂H₄O)ₓH where x varies from about 1 to about 3. Preferred amides are C₈-C₂₀ ammonia amides, monoethanolamides, diethanolamides, and isopropanolamides.

Typically, the nonionic surfactant, when present in the composition, is present in an effective amount, such as from about 0.1% to about 20%, about 0.1% to about 15%, or from about 0.5% to about 10%, by weight.

The surfactant component or formulation hereof may also contain an effective amount of polyhydroxy fatty acid amide surfactant. By “effective amount” is meant that the formulator of the composition can select an amount of polyhydroxy fatty acid amide to be incorporated into the compositions that will improve the cleaning performance of the composition. In general, for conventional levels, the incorporation of about 1%, by weight, polyhydroxy fatty acid amide will enhance cleaning performance.

Such surfactants will typically comprise about 1% weight basis, polyhydroxy fatty acid amide surfactant, preferably from about 3% to about 30%, of the polyhydroxy fatty acid amide. The polyhydroxy fatty acid amide surfactant component comprises compounds of the structural formula:

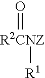

wherein: R¹ is H, C₁-C₄ hydrocarbyl, 2-hydroxy ethyl, 2-hydroxy propyl, or a mixture thereof, preferably C₁ -C₄ alkyl, more preferably C₁ or C₂ alkyl, most preferably C₁ alkyl (i.e., methyl); and R² is a C₅-C₃₁ hydrocarbyl, preferably straight chain C₇-C₁₉ alkyl or alkenyl, more preferably straight chain C₉-C₁₇ alkyl or alkenyl, most preferably straight chain C₁₁-C₁₅ alkyl or alkenyl, or mixtures thereof; and Z is a polyhydroxyhydrocarbyl having a linear hydrocarbyl chain with at least 3 hydroxyls directly connected to the chain, or an alkoxylated derivative (preferably ethoxylated or propoxylated) thereof. Z preferably will be derived from a reducing sugar in a reductive amination reaction; more preferably Z will be a glycityl. Suitable reducing sugars include glucose, fructose, maltose, lactose, galactose, mannose, and xylose. As raw materials, high dextrose corn syrup, high fructose corn syrup, and high maltose corn syrup can be utilized as well as the individual sugars listed above. These corn syrups may yield a mix of sugar components for Z. It should be understood that it is by no means intended to exclude other suitable raw materials. Z preferably will be selected from the group consisting of —CH₂—(CHOH)ₙ—CH₂OH, —CH(CH₂OH)—(CHOH)ₙ₋₁—CH₂OH, —CH₂—(CHOH)₂(CHOR′)(CHOH)—CH₂OH, and alkoxylated derivatives thereof, where n is an integer from 3 to 5, inclusive, and R′ is H or a cyclic or aliphatic monosaccharide. Most preferred are glycityls wherein n is 4, particularly —CH₂—(CHOH)₄—CH₂OH.

R′ can be, for example, N-methyl, N-ethyl, N-propyl, N-isopropyl, N-butyl, N-2-hydroxy ethyl, or N-2-hydroxy propyl.

R²—CO—N< can be, for example, cocamide, stearamide, oleamide, lauramide, myristamide, capricamide, palmitamide, tallowamide, etc.

Z can be 1-deoxyglucityl, 2-deoxyfructityl, 1-deoxymaltityl, 1-deoxylactityl, 1-deoxygalactityl, 1-deoxymannityl, 1-deoxymaltotriotityl, etc.

Methods for making polyhydroxy fatty acid amides are known in the art. In general, they can be made by reacting an alkyl amine with a reducing sugar in a reductive amination reaction to form a corresponding N-alkyl polyhydroxyamine, and then reacting the N-alkyl polyhydroxyamine with a fatty aliphatic ester or triglyceride in a condensation/amidation step to form the N-alkyl, N-polyhydroxy fatty acid amide product. Processes for making compositions containing polyhydroxy fatty acid amides are disclosed, for example, in G.B. Patent Specification 809,060, published Feb. 18, 1959, by Thomas Hedley & Co., Ltd., U.S. Pat. No. 2,965,576, issued Dec. 20, 1960 to E. R. Wilson, and U.S. Pat. No. 2,703,798, Anthony M. Schwartz, issued Mar. 8, 1955, and U.S. Pat. No. 1,985,424, issued Dec. 25, 1934 to Piggott, each of which is incorporated herein by reference.

2. Pearlizing Agent

Pearlescent additives, also known as pearlizing agents, are added to beauty and personal care products such as hair and skin care products to provide a pearly appearance to the products. Micron sized needles or platelets often exhibit this pearly appearance. Materials which exhibit this effect are ethylene glycol mono-and di-stearate, TiO₂ coated mica, bismuth oxychloride, and natural mother of pearl. Many organic materials exhibit this pearlescence provided they can be produced in an appropriate needle or platelet shape.

The pearlizing agent may comprise from about 10% to about 50%, preferably from about 15% to about 40% and most preferably from about 20% to about 25%, by weight based on the total weight of the concentrate.

The pearlizing agent can be selected from the group consisting of, polyethylene glycol mono- and di-stearates, ethylene glycol mono- and distearates, stearic monoethanolamide, glyceryl stearate and mixtures thereof. The preferred agents are polyethylene glycol mono- and distearates, and ethylene glycol mono- and di-stearates. The most preferred pearlizing agents for use are: ethylene glycol mono- and di-stearates.

The fatty acid based member of a typical pearlizing agent, such as those disclosed by U.S. Pat. No. 6,165,955, may be derived from a fatty acid feedstock (which includes free fatty acids, carboxylate salts, fatty mono-, di- and/or tri-glycerides) The fatty acids of the fatty acid based member include combinations of octadecanoic acid and hexadecanoic acid where the majority of these acids include at least about 80% by weight of, more typically at least about 85% by weight, even more typically at least about 90% by weight, e.g., from about 90% to about 100% by weight of the total acid composition used to make the specified pearlizing agent. The ratio of octadecanoic acid to hexadecanoic acid typically ranges from 99:1 to 20:80, more typically from 98:2 to 40:60 and most typically from 95:5 to 60:40, C18:C16. Stearic acid is available commercially in different grades, typically containing at least some portion of palmitic acid, i.e., the saturated fatty acid having one carboxyl group, and a fifteen carbon alkyl tail covalently bonded thereto. For example, stearic acid is available in grades of 37.5% (nominal) and 42.5% (nominal) purity and grades which are 92.5-97.5% (nominal).

The pearlizing agent is most useful as a concentrate with other components, e.g. those other components as described in U.S. Ser. No. 08/542,754, filed Oct. 13, 1995, the disclosure of which is incorporated herein by reference.

3. Alkyl Silicone

Another component of the concentrate of the invention is an alkyl silicone. It is believed that such a silicone can be used to modify the crystal size which in some cases allows for the preparation of high shine and high opacity appearance. In some embodiments the alkyl silicone is a wax. It is believed that the alkyl silicones may be absorbed onto the surface or incorporated into the pearlizing agent, such as EGDS, crystal, thereby altering the appearance of the concentrate and personal care product made therefrom.

Generally, alkyl silicones are used as conditioning agents (occlusive, emollient, or anti-static agents) in personal care formulations. Additional benefits may include thickening for non-aqueous systems.

It is believed, while not being limited by the theory, that the alkyl silicones are useful in the present invention because the alkyl chain can affect the structure and/or growth of the pearl crystals, therefore it is preferable to have an alkyl chain which would be capable of association with the “stearate” in ethylene glycol distearate. Thus, typically, the alkyl chains are C₁₂-C₄₅, preferably, C₁₄-C₂₄ and more preferably C₁₆-C₂₂. Representative exemplary alkyl silicones include: Behenoxy Dimethicone, C₂₀₋₂₄ Alkyl Dimethicone, C₂₄₋₂₈ Alkyl Dimethicone, C₃₀₋₄₅ Alkyl Dimethicone, C₂₀₋₂₄ Alkyl Methicone, C₂₄₋₂₈ Alkyl Methicone, C₃₀₋₄₅ Alkyl Methicone, Cetearyl Methicone, Cetyl Dimethicone, Cetyl Dimethicone Copolyol, Dimethicone Copolyol Beeswax, Dimethicone Copolyol Behenate, Dimethicone Copolyol Isostearate, Dimethicone Copolyol Shea Butter, Dimethicone Copolyol Stearate, Dimethicone Propylethylenediamine Behenate, Dimethiconol Beeswax, Dimethiconol Behenate, Dimethiconol Isostearate, Dimethiconol Stearate, Lauryl Dimethicone, Lauryl Methicone, Laurylmethicone Copolyol, Stearamidopropyl Dimethicone, Stearoxy Dimethicone, Stearyl Aminopropyl Methicone, Stearyl Dimethicone, and Stearyl Methicone. Additional exemplary silicones can be found in International Cosmetic Ingredient Dictionary and Handbook, Volume 2, pages 1707-1709.

Typical alkyl silicones are shown below:

Stearyl Dimethicone

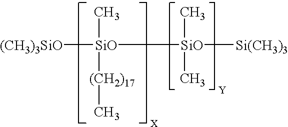

Behenoxy Dimethicone

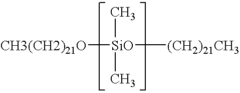

C₂₄₋₂₈ Alkyl Dimethicone

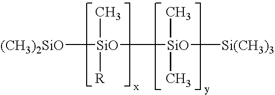

Cetyl Dimethicone

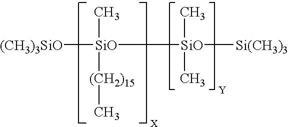

Lauryl Dimethicone

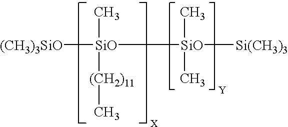

In some embodiments of this invention, dimethiconol stearate (Mirasil Wax S) and dimethiconol benehate (Mirasil Wax B) are preferred.

4. Water

The remainder of the concentrate of the invention is water, preferably deionized. Generally, water may be included in an amount of from about 20% to about 70%, preferably from about 30% to about 60%, and most preferably from about 40% to about 55% based on the total weight of the concentrate.

5. Other Additives

Beauty care and personal care products, such as shampoos and soaps for hand and/or body wash, of the present invention contain adjunct ingredients. Additional background on such products is provided by PCT application serial number PCT/US98/04474, filed Mar. 6, 1998 and published as WO 98/38973, incorporated herein by reference in its entirety.

Optional components can be utilized in the concentrates of the present invention as a convenient means of incorporation into beauty and personal care products. Such conventional optional ingredients are well known to those skilled in the art, e.g., preservatives such as benzyl alcohol, methyl paraben, propyl paraben and imidazolidinyl urea; thickeners and viscosity modifiers such as block polymers of ethylene oxide and propylene oxide, e.g. ANTAROX F-88 (Rhodia, Inc.), polyvinyl alcohol, and ethyl alcohol; pH adjusting agents such as citric acid, succinic acid, phosphoric acid, sodium hydroxide, sodium carbonate; perfumes; dyes; and sequestering agents such as disodium ethylenediamine tetra-acetate. Such agents generally are used individually at levels of from 0% to about 5%, preferably from about 0.01% to about 2.0% by weight of the concentrate.

Other additional optional additives include electrolytes. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride and sodium or ammonium sulfate, in an amount ranging from about 0.1-10% of the concentrate, preferably between about 0.5-5% when present.

6. Method of Making the Concentrate

Also the present invention provides a method for producing a mild, cold pearlizing concentrate comprising:

providing a mixture of water, at least one surfactant, at least one alkyl silicone; and at least one pearlizing agent,

maintaining the mixture at a temperature higher than the melting point of all ingredients of the mixture.

In one embodiment the method for producing the mild, cold pearlizing concentrate comprises:

(a) heating the at least one surfactant and at least a portion of the water of the mixture to a temperature higher than the melting point of all ingredients of the mixture;

(b) adding an alkyl silicone to the heated mixture; and

(c) adding a pearlizing agent to the heated mixture.

In the heating step, the mixture is heated to a temperature higher than the melting point of all the ingredients currently in the mixture or to be added to the mixture in forming the concentrate. Then the alkyl silicone and pearlizing agent are added to the heated mixture in any order. The combined ingredients are maintained at the temperature for a time sufficient to melt/dissolve all the ingredients. Afterwards, the mixture is cooled to create the appropriate crystal size and appearance.

II. Compositions For Personal Care Products

The cold pearlizing concentrate of the present invention can be specifically formulated into a wide variety of personal care and beauty care products. These products can be formulated by one skilled in the art utilizing conventional methods of production. The pearlizing concentrate imparts a specific appearance, in some cases a high luster pearlescence and sheen to the products. Generally, the shampoos and soaps of the present invention can be made by merely mixing the beauty and/or personal care product together with the concentrate at room temperature.

Typical personal care products include shampoos, hand soap, liquid soap, body wash, facial cleansers, baby cleansers, children's cleansers, and bubble bath, and may be formulated as described by U.S. Pat. No. 5,560,879, herein incorporated by reference in its entirety.

Such formulated shampoo and soap systems utilizing the cold pearlizing concentrate of the present invention can contain a variety of non-essential optional components suitable for rendering such compositions more acceptable. Such conventional optional ingredients are well known to those skilled in the art.

Personal care compositions in accordance with the invention may also additional contain silicone compounds.

The shampoo or other personal care cleansing products will generally include an additional surfactants. These include anionic, cationic, nonionic surfactants, amphoteric surfactants, zwitterionic surfactants. Examples of anionic surfactants are described in U.S. Pat. No. 5,573,709, the entire disclosure of which is incorporated by reference. Preferred anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocyl sarcosine, ammonium cocyl sulfate, ammonium lauroyl sulfate, sodium cocyl sulfate, sodium lauroyl sulfate, potassium cocyl sulfate, potassium lauryl sulfate, monoethanolamine cocyl sulfate, sodium tridecyl benzene sulfonate, or sodium dodecyl benzene sulfonate.

Some examples of nonionic surfactants which can be used in the shampoo composition or other personal care cleansing products include those broadly defined as compounds produced by the condensation of alkylene oxide groups (hydrophilic in nature) with an organic hydrophobic compound, which may be aliphatic or alkyl aromatic in nature.

Zwitterionic surfactants, for use in a shampoo or other personal care products are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. A general formula for these compounds is: found in U.S. Pat. No. 5,573,709, which is incorporated herein by reference, wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants, such as betaines can also useful in the personal care products of the invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the 1 like; amidobetaines and amidosulfobetaines, wherein the RCONH(CH₂) 3 radical is attached to the nitrogen atom of the betaine are also useful in this invention. Preferred betaines for use in the present compositions are cocoamidopropyl betaine, cocobetaine, lauryl amidopropyl betaine, and oleyl betaine.

Examples of amphoteric surfactants which can be used in the personal care compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products sold under the trade name MIRANOL and described in U.S. Pat. No. 2,528,378. Another surfactant optional for use in the personal care compositions of the present invention is cocoamphocarboxy glycinate.

The typical shampoos or other personal care cleansing products of the present invention contain combinations of amphoteric surfactants, zwitterionic surfactants, nonionic surfactants, anionic surfactants, or combinations thereof. A typical shampoo formulation or other personal care cleansing product typically contains from about 0% to about 6% of amphoteric surfactants, about 0% to about 8% of zwitterionic surfactants, from 0% to about 20% of primary anionic surfactants such as ethoxylated alkyl sulfates, alkyl sulfates, alpha-olefin sulfonates, or mixtures thereof, and from about 0% to about 10% of an optional anionic surfactant surfactants, with a typical total surfactant level of from about 6% to about 25%, more preferably from about 10 to about 15%.

The formulated shampoo and soap systems utilizing the cold pearlizing concentrate of the present invention can contain a variety of non-essential optional components suitable for rendering such compositions more acceptable. Such conventional optional ingredients are well known to those skilled in the art, e.g., preservatives such as benzyl alcohol, methyl paraben, propyl paraben and imidazolidinyl urea; cationic surfactants such as cetyl trimethyl ammonium chloride, lauryl trimethyl ammonium chloride, tricetyl methyl ammonium chloride, stearyldimethyl benzyl ammonium chloride, and di(partially hydrogenated tallow)dimethylammonium chloride; thickeners and viscosity modifiers such as block polymers of ethylene oxide and propylene oxide, e.g. ANTAROX F-88 (Rhodia, Inc. Inc.), sodium chloride, sodium sulfate, polyvinyl alcohol, and ethyl alcohol; pH adjusting agents such as citric acid, succinic acid, phosphoric acid, sodium hydroxide, sodium carbonate; perfumes; dyes; conditioning agents such as silicones; active ingredients such as anti-dandruff agents (zinc pyrithion); vitamins such as Vitamin B, Vitamin E Acetate; and sequestering agents such as disodium ethylenediamine tetra-acetate. Such agents generally are used individually at levels of from about 0.01% to about 10%, preferably from 0.5% to about 5.0% by weight of the composition depending on the desired properties of the formulation.

III. Examples

The following examples are provided to better describe and define the concentrate and personal care compositions of the present invention. They are for illustrative purposes only, and it is realized that changes and variations may be made with respect to these compositions that are not shown below. Such changes that do not materially alter the compositions formulation or function are still considered to fall within the spirit and scope of the invention as recited by the claims that follow. As used in this specification and in the appended claims, all parts, percentages, ratios and the like are by weight of active ingredient, e.g., active surfactant, unless otherwise apparent, in context, as is the case in the examples.

A typical concentrate (Example A) formulation in accordance with the invention comprises 30% by weight of MIRACARE LAC116 (containing water, ammonium laureth sulfate, cocamide MEA) available from Rhodia, Inc., 20% EGDS, 3% dimethiconol stearate (MIRASIL WAX S), and the remainder being water.

Examples A through D of TABLE I are preferably formed by the following method. The water and the MIRACARE LAC116 are mixed together and heated to approximately 80° C. Once the temperature is reached, the alkyl silicone and pearlizing agent, i.e., EGDS, is added, and the temperature is maintained and stirred until the entire mixture, including the EGDS crystals, has dissolved, often approximately 1 hour or more. Cooling to 60° C. can be completed as quickly as possible, cooling from 60° C. to 40° C. should be more carefully controlled to create the appropriate crystal size and appearance. The Comparative Example of TABLE I can be made in a similar fashion, without the inclusion of the alkyl silicone.

The Brookfield viscosity in Table I was measured at 25° C., #4 spindle at 30 rpm for 30 seconds. The particle size in Table I was measured using the Coulter LS 130 Particle Size Analyzer micro-volume module in a solute of water.

TABLE I

| | Example | Example | Example | Example | Comparative |
|---|---|---|---|---|---|
| | A | B | C | D | Example I |
| | (% by | (% by | (% by | (% by | (% by |
| | weight) | weight) | weight) | weight) | weight) |
| MIRACARE LAC116 | 29% | 30% | 29% | 29% | 30% |
| (water, ammonium laureth | | | | | |
| sulfate, cocamide MEA) | | | | | |
| Ammonium Chloride | 1% | 1% | 1% | 1% | 1% |
| MIRASIL WAX S | 3% | 0.5% | 6% | — | — |
| (Dimethiconol Stearate) | | | | | |
| MIRASIL WAX B | — | — | — | 3% | — |
| (Dimethiconol Behenate) | | | | | |
| EGDS | 19% | 20% | 19% | 19% | 20% |
| Water | 48% | 48.5% | 45% | 48% | 49% |
| Viscosity (centipoise) | 3000-3200 | | | | 4000 |
| Median Particle Size (microns) | 12.9 | 14.2 | 19.1 | 13.6 | 10.7 |

Example A was diluted with water and studied against the Comparative Example I, also diluted in water, to make Example E and Comparative Example II, respectively. They exhibited properties listed in TABLE II.

Examples B through D, when incorporated in basic shampoo formulations (Examples F-H, respectively) exhibited the properties presented in TABLE III, when compared to a conventional composition (Comparative Example III), i.e., without any alkyl silicone component.

TABLE II

| | Example E | Comparative Example II |
|---|---|---|
| | (% by weight) | (% by weight) |
| Water | 98% | 98% |
| Pearlizing | | |
| Concentrate of: | | |
| Example A | 2% | — |
| Comparative | — | 2% |
| Example I | | |
| Appearance: | | |
| Opacity | Low/Moderate | Low |

| | |
|---|---|
| Shine | Same for both |

TABLE III

| | Example | Example | Example | Comparative |
|---|---|---|---|---|
| | F | G | H | Example III |
| | (% by weight) | (% by weight) | (% by weight) | (% by weight) |
| Miracare LAC116 | 20% | 20% | 20% | 20% |
| Sodium Chloride | 1% | 1% | 1% | 1% |
| Water | 75% | 75% | 75% | 75% |
| Pearlizing | | | | |
| Concentrate of: | | | | |
| Example B | 4% | — | — | — |
| Example C | — | 4% | — | — |
| Example D | — | — | 4% | — |
| Comparative | — | — | — | 4% |
| Example I | | | | |
| Appearance: | | | | |
| Opacity | Low/Moderate | Low | Low | Low |
| Shine | Moderate | Moderate/High | Moderate/High | Moderate |
| Pearl Effect | Silky Shine | Metallic Shine | Metallic Shine | Silky Shine |

The appearance values in TABLE II were determined by visual assessment relative to the other examples in the given TABLE II. The appearance values in TABLE II were determined after adding 4% of the cold pearlizing concentrate to a shampoo formulation. Thus, the inventors believe, but do not wish to be bound by the theory, the particle size data shows that the inclusion of the alkyl silicone will affect the crystal size and therefore the crystal appearance by the association of the alkyl group on the silicone with the alkyl group in the pearlizing agent during crystallization. This can affect the appearance of the resulting crystals by increasing the pearl effect, shine and/or increasing the opacity.

The appearance values in TABLE III were determined by visual assessment relative to the other examples in the given TABLE III.

Other studies have shown that the order of adding the EGDS and alkyl silicone does not matter. In other words, as long as the concentrate is heated to above the melting point of the alkyl silicone, very little difference in resulting properties is seen if the alkyl silicone is added before or after the EGDS.

A pearlescent liquid hand soap including the concentrate of the invention could be prepared as follows:

TABLE IV
EXAMPLE I

| | |
|---|---|
| RHODACAL A-246-L (Rhodia, Inc.) | 23.4 |
| (Sodium α olefin sulfonate) | |
| Pearlizing Concentrate of Example B | 12.0 |
| Citric Acid (to pH 6.5-7.0) | q.s. |
| Perfume, Dye, Preservative | q.s. |
| Sodium chloride | q.s. |
| Water, deionized | 60.6 |

To formulate Example I, the water is charged into a mixing vessel and the active ingredients are slowly mixed at room temperature until the mixture becomes uniform in appearance and texture. Citric acid (50%) is then added in an amount sufficient to adjust the pH to 6.0. The composition is then fragranced with a suitable perfume as desired and colored with an appropriate FD&C dye.

The following ingredients and their respective amounts can be combined to produce an ultra-mild body shampoo in accordance with the invention.

TABLE V
EXAMPLE J

| | |
|---|---|
| RHODAPON SB-8208/s | 17.2 |
| (Rhodia, Inc.) | |
| GERPOPON SBFA 30 (Rhodia, Inc.) | 4.2 |
| (disodium laureth (3EO) sulfate sulfo | |
| succinate, 30% active) | |
| Pearlizing Concentrate of Example D | 8.5 |
| MIRATINE BET C-30 (Rhodia, Inc.) | 7.7 |
| (cocamido propyl betaine, 30% active) | |
| Perfume, Dye, Perservative | q.s. |
| Sodium Chloride | q.s. |
| Water, deionized | 62.4 |

A second exemplary pearlescent mild conditioning shampoo may be prepared as follows:

TABLE VI
EXAMPLE K

| | |
|---|---|
| Shampoo Blend | 30.0 |
| Sodium Laureth Sulfate | 5.0 |
| Sodium Cocoamphoacetate (40%) | 3.0 |
| Cocoamide DEA | 2.0 |
| Dimethicone Emulsion | 6.0 |
| Pearlizing Concentrate of Example A | 4.0 |
| Preservative | q.s. |
| Dye | q.s. |
| Fragrance | q.s. |
| Sodium Chloride | q.s. |
| Deionized Water | q.s. to 100 |

A shampoo blend of sodium methyl cocoyl taurate, cocoamidopropyl betaine, cocoamide DEA and glycerine was mixed with sodium laureth sulfate (RHODAPEX 3N70, Rhodia, Inc.), cocoamphoacetate (MIRANOL Ultra C-32, Rhodia, Inc.) cocoamide DEA (ALKAMIDE DC 212/S, Rhodia, Inc.), dimethicone (MIRASIL DM-E, Rhodia, Inc.), and the remaining conventional shampoo ingredients may also be prepared, to which, the cold pearlizing concentrate of Example A can be added to with agitation at room temperature.

It should be apparent that embodiments other than those specifically described above may come within the spirit and scope of the present invention. Hence, the present invention is not limited by the above description, but rather is defined by the claims appended hereto.

We claim:

1. A mild cold pearlizing concentrate comprising a mixture of:
(a) a surfactant formulation comprising at least one surfactant-
(b) at least one pearlizing agent;
(c) at least one alkyl silicone; and
(d) water.

2. The concentrate of claim 1, wherein the alkyl silicone is a silicone wax.

3. The concentrate of claim 1, wherein the alkyl silicone comprises at least one C₁₂-C₄₅ chain.

4. The concentrate of claim 1, wherein the alkyl silicone comprises at least one C₁₄-C₂₄ chain.

5. The concentrate of claim 1, wherein the alkyl silicone comprises at least one C₁₆-C₂₂ chain.

6. The concentrate of claim 3, wherein the alkyl silicone comprises at least one stearate or benehate chain.

7. The concentrate of claim 1, wherein the alkyl silicone is at least one selected from the group consisting of Behenoxy Dimethicone, C₂₀₋₂₄ Alkyl Dimethicone, C₂₄₋₂₈ Alkyl Dimethicone, C₃₀₋₄₅ Alkyl Dimethicone, C₂₀₋₂₄ Alkyl Methicone, C₂₄₋₂₈ Alkyl Methicone, C₃₀₋₄₅ Alkyl Methicone, Cetearyl Methicone, Cetyl Dimethicone, Cetyl Dimethicone Copolyol, Dimethicone Copolyol Beeswax, Dimethicone Copolyol Behenate, Dimethicone Copolyol Isostearate, Dimethicone Copolyol Shea Butter, Dimethicone Copolyol Stearate, Dimethicone Propylethylenediamine Behenate, Dimethiconol Beeswax, Dimethiconol Behenate, Dimethiconol Isostearate, Dimethiconol Stearate, Lauryl Dimethicone, Lauryl Methicone, Laurylmethicone Copolyol, Stearamidopropyl Dimethicone, Stearoxy Dimethicone, Stearyl Aminopropyl Methicone, Stearyl Dimethicone, and Stearyl Methicone.

8. The concentrate of claim 1, wherein the alkyl silicone is at least one selected from the group consisting of dimethiconol stearate and dimethiconol benehate.

9. The concentrate of claim 1, wherein the pearlizing agent comprises at least one selected from the group consisting of polyethylene glycol monostearate, polyethylene glycol distearate, glyceryl stearate, ethylene glycol monostearate and ethylene glycol distearate.

10. The concentrate of claim 1, wherein the pearlizing agent comprises at least one selected from the group consisting of ethylene glycol monostearate and ethylene glycol distearate.

11. The concentrate of claim 1, wherein the at least one surfactant comprises at least one selected from the group consisting of amphoteric surfactants, zwitterionic surfactants, anionic surfactants, nonionic surfactants, and cationic surfactants.

12. The concentrate of claim 11, wherein the surfactant formulation comprises at least one nonionic surfactant selected from the group consisting of alkanolamides, amine oxides, alkyl ethoxylates, alkanoyl glucose amide, and alkyl phenol ethoxylates.

13. The concentrate of claim 11, wherein the surfactant formulation comprises at least one surfactant is selected from the group consisting of alkyl betaines, amidopropyl betaines, sultaines, propionates, and amphoacetates.

14. The concentrate of claim 11, wherein the surfactant formulation comprises at least one anionic surfactant selected from the group consisting of alkyl sulfates, alkyl ether sulfates, sulfosuccinates, taurates, ether carboxylates, fatty soaps, isethionates, alkyl phosphates, linear alkylbenzene sulfonates, alpha olefin sulfonates, paraffin sulfonates, and alkyl ester sulfonates.

15. The concentrate of claim 1, comprising, by weight on a total concentrate basis:

| | |
|---|---|
| the surfactant | about 10-35%; |
| the pearlizing agent | about 10-50%; |
| the alkyl silicone | about 0.1-10%; and |
| water | remainder. |

16. The concentrate of claim 15, comprising, by weight on a total concentrate basis:

| | |
|---|---|
| the surfactant | about 15-30%; |
| the pearlizing agent | about 15-30%; |
| the alkyl silicone | about 0.5-5%; and |
| water | remainder. |

17. The concentrate of claim 15, comprising, by weight on a total concentrate basis:

| | |
|---|---|
| the surfactant | about 18-25%; |
| the pearlizing agent | about 20-25%; |
| the alkyl silicone | about 2-4%; and |
| water | remainder. |

18. The concentrate of claim 1, wherein the concentrate comprises about 0.1-10% alkyl silicone by weight on a total concentrate basis.

19. The concentrate of claim 1, wherein the concentrate comprises about 2-4% alkyl silicone by weight on a total concentrate basis.

20. The concentrate of claim 1, further comprising at least one additive, in an amount of 0-10% by total weight of the concentrate, selected from the group consisting of electrolytes, preservatives, thickeners, viscosity modifiers, pH adjusting agents, and sequestering agents.

21. The concentrate of claim 20, wherein the electrolyte comprises at least one selected from the group consisting of sodium chloride, ammonium chloride, sodium sulfate, ammonium sulfate.

22. A method for producing a mild, cold pearlizing concentrate comprising:
providing a mixture of water, at least one surfactant, at least one alkyl silicone; and at least one pearlizing agent,
maintaining the mixture at a temperature higher than the melting point of all ingredients of the mixture.

23. The method of claim 22, comprising:
(a) heating the at least one surfactant and at least a portion of the water of the mixture to a temperature higher than the melting point of all ingredients of the mixture;
(b) adding the at least one alkyl silicone to the heated mixture; and
(c) adding the at least one pearlizing agent to the heated mixture.

24. The method of claim 23, comprising maintained the mixture at the temperature for a time sufficient to melt/dissolve all the ingredients, and then cooling the heated mixture to create the desired crystal size and appearance.

25. The method of claim 23, wherein step (c) is performed before step (b).

26. A personal care product comprising the concentrate of claim 1.

27. The personal care product of claim 26, wherein the product is a cleaning product.

27. The personal care product of claim 26, wherein the cleaning product is selected from the group consisting of a shampoo, hand soap, liquid soap, body wash, facial cleanser, baby cleanser, children's cleanser, and bubble bath.

28. A personal care product of claim 25, where the personal care product is a conditioning/moisturizing product.

29. The personal care product of claim 28, wherein the conditioning/moisturizing product is a hair conditioner, a lotion, or a cream.
